(19) United States
(12) Patent Application Publication
(10) Pub. No.: US 2015/0195679 A1
(43) Pub. Date: Jul. 9, 2015

(54) PORTABLE DEVICE

(71) Applicant: Seiko Epson Corporation, Shinjuku-ku, Tokyo (JP)

(72) Inventors: Eiji Miyasaka, Okaya-shi (JP); Kazuto Mogami, Suwa-shi (JP); Takahiro Kunishige, Shiojiri-shi (JP)

(21) Appl. No.: 14/409,927

(22) PCT Filed: Jun. 19, 2013
(86) PCT No.: PCT/JP2013/003815

§ 371 Date: Dec. 19, 2014

(30) Foreign Application Priority Data
Jun. 22, 2012 (JP) 2012-140310

(51) Int. Cl. H04W 4/02; G01P 7/00; G01S 19/49; G01C 22/00; A63B 24/00; A61B 5/11; A61B 5/00; G01P 15/02; G01S 19/19

(52) U.S. Cl. CPC H04W 4/027; G01P 15/02; G01P 7/00; G01S 19/49; G01S 19/19; A63B 24/00; A61B 5/1112; A61B 5/1122; A61B 5/7242; A61B 5/681; G01C 22/006; A61B 2562/0219

(57) ABSTRACT

A portable device includes an acceleration sensor capable of detecting accelerations in three-axis directions. When a GPS radio wave is receivable by a GPS module, the portable device stores, using an MCU, a correlation between a body vibration frequency detected by the acceleration sensor and speed information grasped by processing a signal included in the GPS radio wave in a flash ROM. When a reception state of the GPS radio wave does not satisfy a predetermined standard, the portable device estimates running speed using the MCU on the basis of the body vibration frequency detected by the acceleration sensor 38 and the correlation stored in the flash ROM.

The application claims the benefit of Japanese Patent Application No. 2012-140310 filed Jun. 22, 2012 and PCT International Application No. PCT/JP2013/003815, filed Jun. 19, 2013, which are hereby incorporated by reference herein in their entirety.

### BACKGROUND OF THE INVENTION

1. Technical Field

The present invention relates to a portable device including a reception module configured to receive radio waves from position information satellites and an acceleration sensor.

2. Related Art

In a running watch that displays speed information and the like grasped by processing signals from position information satellites such as GPS (Global Positioning System) satellites, when radio wave information from the position information satellites cannot be received in a tunnel or the like, the problem is how to display running speed.

As described in PTL 1, there has been proposed a method of using an acceleration sensor separate from a running watch and, when radio wave information from position information satellites cannot be received, learning a running ability of a user on the basis of an output signal from the acceleration sensor and estimating running speed of the user.

### SUMMARY OF INVENTION

However, in the method of Cited Literature 1, since the user needs to wear a foot sensor, a wrist sensor, and a chest sensor in the respective regions, there is a problem in that the wearing of the sensors is troublesome. There is also a problem in that battery replacement is necessary for these sensors and, when old batteries are used, the user fails in data communication and recording because of a voltage shortage.

Further, concerning the wrist sensor, since motions of arms during running are various depending on users, it is difficult to accurately catch a waveform peak, causing deteriorate in accuracy of an output value. Since there are also runners who do not swing the arms, there is a problem in that an accurate waveform of arm swing cannot be acquired.

An advantage of some aspects of the invention is to provide a portable device that has no trouble of sensor wearing and can surely and accurately perform speed calculation irrespective of what kind of a user uses the portable device and even when signals from position information satellites cannot be received.

An aspect of the invention is directed to a portable device including: a radio-wave receiving unit configured to receive a radio wave from a position information satellite; a detecting unit capable of detecting a body vibration frequency in a direction in which the body of a user moves up and down during running; a display unit configured to display running information; a specifying unit configured to specify, when a reception state of the radio wave satisfies a predetermined standard, a correlation between the body vibration frequency detected by the detecting unit and speed information grasped by processing a signal included in the radio wave; and an estimating unit configured to estimate, when the reception state of the radio wave does not satisfy the predetermined standard, running speed or a running pace on the basis of the body vibration frequency detected by the detecting unit and the correlation specified by the specifying unit.

In the portable device, when the reception state of the radio wave from the position information satellite satisfies the predetermined standard, the correlation between the body vibration frequency detected by the detecting unit and the speed information grasped by processing the signal included in the radio wave is specified by the specifying unit. When the reception state of the radio wave does not satisfy the predetermined standard, the estimating unit estimates running speed or a running pace on the basis of the body vibration frequency detected by the detecting unit and the correlation specified by the specifying unit.

Therefore, according to the aspect of the invention, since the detecting unit is provided in the portable device, there is no trouble of wearing sensors in regions of the body other than the portable device. Since running speed or a running pace is estimated on the basis of the body vibration frequency in the direction in which the body of the user moves up and down during running, it is possible to surely and accurately estimate running speed or a running pace irrespective of a step and an exercise capacity of the user.

The portable device of the aspect of the invention may be configured such that the detecting unit includes an acceleration sensor configured to detect accelerations in three axis directions of an X axis, a Y axis, and a Z axis and detect, as the body vibration frequency, a frequency of vibration in a direction of an axis closest to the gravity direction among frequencies of vibrations obtained by the acceleration sensor. According to this configuration, since the detecting unit includes the tri-axis acceleration sensor, it is possible to obtain accelerations concerning the respective X, Y, and Z axes.

Incidentally, an axis reflecting vibration in the up down direction most is different depending on a form of wearing of the portable device by the user. According to the configuration described above, the frequency of the vibration of the axis closest to the gravity direction is specified as the body vibration frequency in the form of wearing the portable device by the user. Therefore, it is possible to estimate running speed or a running pace on the basis of the body vibration frequency in the direction in which the body of the user moves up and down during running and it is possible to surely and accurately estimate running speed or a running pace irrespective of a step and an exercise capacity of the user.

The portable device of the aspect of the invention may be configured such that the detecting unit includes an acceleration sensor configured to detect accelerations in three axis directions of an X axis, a Y axis, and a Z axis, combine waveforms of vibrations of the respective axes obtained by the acceleration sensor, and detect a frequency of a combined waveform as the body vibration frequency. By configuring the portable device in this way, it is possible to estimate running speed or a running pace on the basis of the body vibration frequency in the direction in which the body of the user moves up and down during running and it is possible to surely and accurately estimate running speed or a running pace irrespective of a step and an exercise capacity of the user.

The portable device may be configured such that the detecting unit includes an acceleration sensor configured to detect accelerations in three axis directions of an X axis, a Y axis, and a Z axis and detect, as the body vibration frequency, a frequency of a waveform of an axis having largest amplitude among waveforms of vibrations of the respective axes obtained by the acceleration sensor. By configuring the portable device in this way, it is possible to estimate running speed or a running pace on the basis of the body vibration frequency in the direction in which the body of the user moves up and down during running and it is possible to surely and accurately estimate running speed or a running pace irrespective of a step and an exercise capacity of the user.

The portable device may be configured such that the specifying unit specifies, as a linear expression, the correlation between the body vibration frequency and the speed information grasped by processing the signal included in the radio wave, and the estimating unit estimates running speed or a running pace from the body vibration frequency on the basis of the linear expression. By configuring the portable device in this way, it is possible to reduce a data amount that should be recorded as the correlation. When the reception state of the radio wave does not satisfy the predetermined standard, it is possible to surely and accurately estimate running speed or a running pace.

The portable device may be configured such that the specifying unit generates a table for specifying the correlation between the body vibration frequency and the speed information grasped by processing the signal included in the radio wave, and the estimating unit estimates running speed or a running pace from the body vibration frequency referring to the table. By configuring the portable device in this way, when the reception state of the radio wave does not satisfy the predetermined standard, it is possible to surely and accurately estimate running speed or a running pace.

The portable device may be configured such that, when the reception state of the radio wave satisfies the predetermined standard, a running distance is calculated from the speed information grasped by processing the signal included in the radio wave, and when the reception state of the radio wave does not satisfy the predetermined standard, the running distance is calculated on the basis of the running speed and time or the running pace and time estimated by the estimating unit. By configuring the portable device in this way, it is possible to accurately calculate the running distance even when the reception state of the radio wave does not satisfy the predetermined standard.

The portable device may be configured such that, when the reception state of the radio wave satisfies the predetermined standard, a running distance is calculated from the position information grasped by processing the signal included in the radio wave, and when the reception state of the radio wave does not satisfy the predetermined standard, the running distance is calculated on the basis of the running speed and time or the running pace or time estimated by the estimating unit. By configuring the portable device in this way, it is possible to accurately calculate the running distance even when the reception state of the radio wave does not satisfy the predetermined standard.

### BRIEF DESCRIPTION OF DRAWINGS

FIG. 1 is an overall diagram of a GPS system including a GPS running watch according to an embodiment of the invention.

FIG. 2 is a plan view of the GPS running watch.

FIG. 3 is a block diagram showing a circuit configuration of the GPS running watch.

FIG. 4 is a diagram for explaining an error of a step detected using a separate acceleration sensor attached to a shoe.

FIG. 5 is a graph showing a relation between a body vibration frequency and running speed.

FIG. 6 is a diagram for explaining processing at GPS reception time and at GPS reception impossible time in the embodiment of the invention.

FIG. 7 is a flowchart for explaining calculation processing for speed and a distance in the embodiment of the invention.

FIG. 8 is a graph showing output waveforms of axes of the acceleration sensor.

FIG. 9 is graphs of relations between body vibration frequencies and running speeds calculated concerning various users.

FIG. 10 is a diagram for explaining a difference between measuring methods for a running distance during running in a curved tunnel.

FIG. 11 is a diagram showing directions of the axes of the acceleration sensor of the GPS running watch 100 worn on the arm.

FIG. 12 is a graph showing vibrations in the axis directions caused during normal running and when a user looks at a screen of the GPS running watch.

FIG. 13 is a graph obtained by calculating a relation between a body vibration frequency and running speed of a runner A.

FIG. 14 is a graph showing a linear expression calculated from data shown in FIG. 13.

FIG. 15 is a graph obtained by calculating a relation between a body vibration frequency and running speed of a runner B.

FIG. 16 is a graph showing a linear expression calculated from data shown in FIG. 15.

FIG. 17 is a graph obtained by calculating a relation between a body vibration frequency and running speed of a runner C.

FIG. 18 is a graph showing a linear expression calculated from data shown in FIG. 17.

FIG. 19 is a graph showing a quadratic equation calculated from the data shown in FIG. 13.

FIG. 20 is a graph showing a quadratic expression calculated from the data shown in FIG. 15.

FIG. 21 is a graph showing a quadratic expression calculated from the data shown in FIG. 17.

### DESCRIPTION OF EXEMPLARY EMBODIMENTS

A preferred embodiment of the invention is explained in detail below with reference to the accompanying drawings and the like. In the figures, dimensions and scales of units are set different from actual ones as appropriate. Since the embodiment explained below is a preferred specific example of the invention, technically preferable various limitations are given. However, the scope of the invention is not limited to these forms as long as, in the following explanation, there is no particular description indicating that the invention is limited.

### A: Mechanical Configuration of a GPS Running Watch

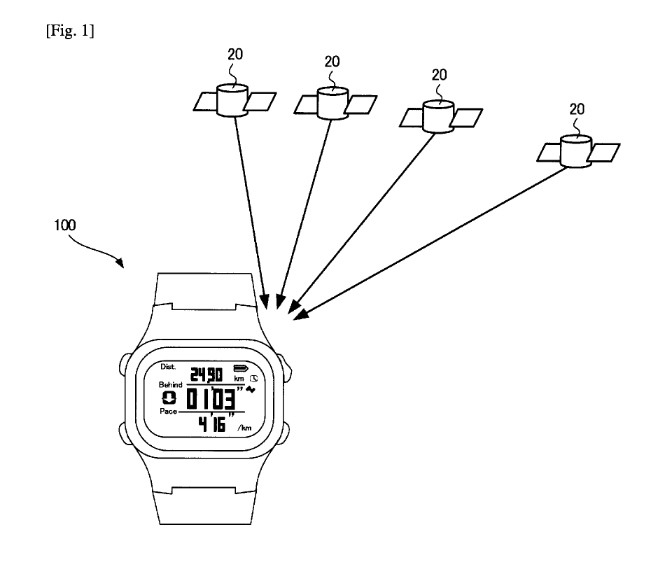

FIG. 1 is an overall diagram of a GPS system including a GPS running watch 100 used as a portable device according to an embodiment of the invention. The GPS running watch 100 is a portable device that displays speed information and the like grasped by processing radio waves (radio signals) from GPS satellites 20. The GPS running watch 100 displays time on a surface (hereinafter referred to as “front surface”) on the opposite side of a surface in contact with the arm (hereinafter referred to as “rear surface”).

The GPS satellites 20 are position information satellites that revolve on a predetermined orbit in the sky of the earth. The GPS satellites 20 transmit a navigation message to the ground while superimposing the navigation message on a 1.57542 GHz radio wave (a L1 wave). In the following explanation, the 1.57542 GHz radio wave on which the navigation message is superimposed is referred to as “satellite signal”. The satellite signal is a right-handed circularly polarized wave.

At present, about thirty-one GPS satellites 20 (in FIG. 1, only four GPS satellites among the about thirty-one GPS satellites are shown) are present. In order to identify from which of the GPS satellites 20 the satellite signal is transmitted, the respective GPS satellites 20 superimpose 1023 chip (1 ms period) peculiar patterns called C/A codes (Coarse/Acquisition Codes) on satellite signals. The C/A codes have chips of +1 or −1 and look like random patterns. Therefore, it is possible to detect a C/A code superimposed on the satellite signal by calculating a correlation between the satellite signals and the patterns of the C/A codes.

The GPS satellites 20 are mounted with atomic clocks. Extremely accurate time information (hereinafter referred to as “GPS time information”) measured by the atomic clocks is included in the satellite signal. A slight time error of atomic clocks mounted on the GPS satellites 20 is measured by a control segment on the ground. A time correction parameter for correcting the time error is also included in the satellite signal. The GPS running watch 100 receives the satellite signal transmitted from one GPS satellite 20 and corrects internal time to accurate time using the GPS time information and the time correction parameter included in the satellite signal.

Satellite orbit information indicating positions on an orbit of the GPS satellites 20 is also included in the satellite signal. The GPS running watch 100 can perform a positioning calculation using the GPS time information and the satellite orbit information. The positioning calculation is performed on the premise that a certain degree of an error is included in the internal time of the GPS running watch 100. That is, in addition to x, y, and z parameters for specifying a three-dimensional position of the GPS running watch 100, a time error is also an unknown number. Therefore, in general, the GPS running watch 100 receives satellite signals respectively transmitted from four or more GPS satellites and performs the positioning calculation using GPS time information and satellite orbit information included in the satellite signals.

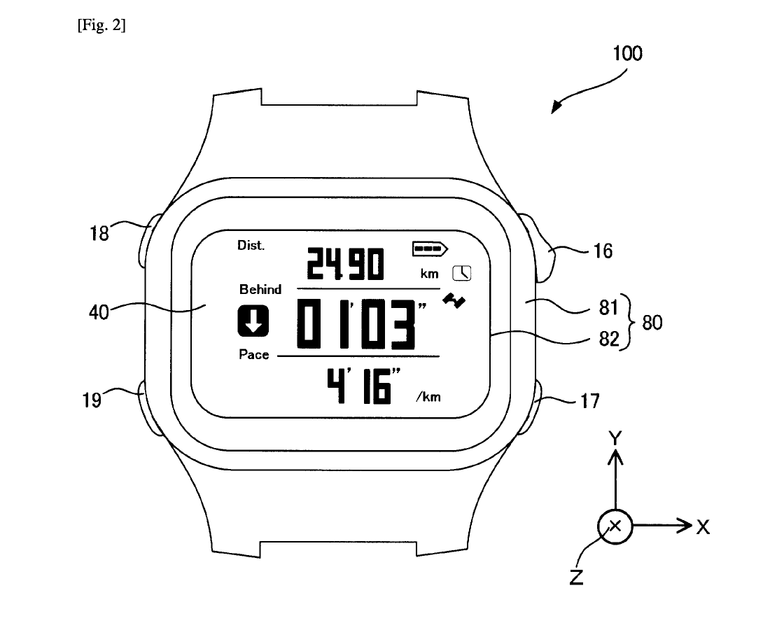

FIG. 2 is a plan view of the GPS running watch 100. As shown in FIG. 2, the GPS running watch 100 includes an armor case 80. The armor case 80 is configured by fitting a cover glass 82 formed of glass or plastic in a case 81 formed of plastic. In this embodiment, the armor case is configured by two components. However, the armor case may be configured by one component. Alternatively, the armor case may be configured by three components using a back cover. A liquid crystal panel 40 is arranged under the cover glass 82. The liquid crystal panel 40 is configured to display running information such as running speed, a running distance, a running time, a running pace (e.g., a required time (minutes) per 1 km), a pitch (the number of steps per one minute), and the number of steps.

The GPS running watch 100 can be switched to a running mode for displaying running speed and the like, a time display mode for displaying time, and the like by manually operating operation buttons 16 to 19.

### B: Circuit Configuration of the GPS Running Watch

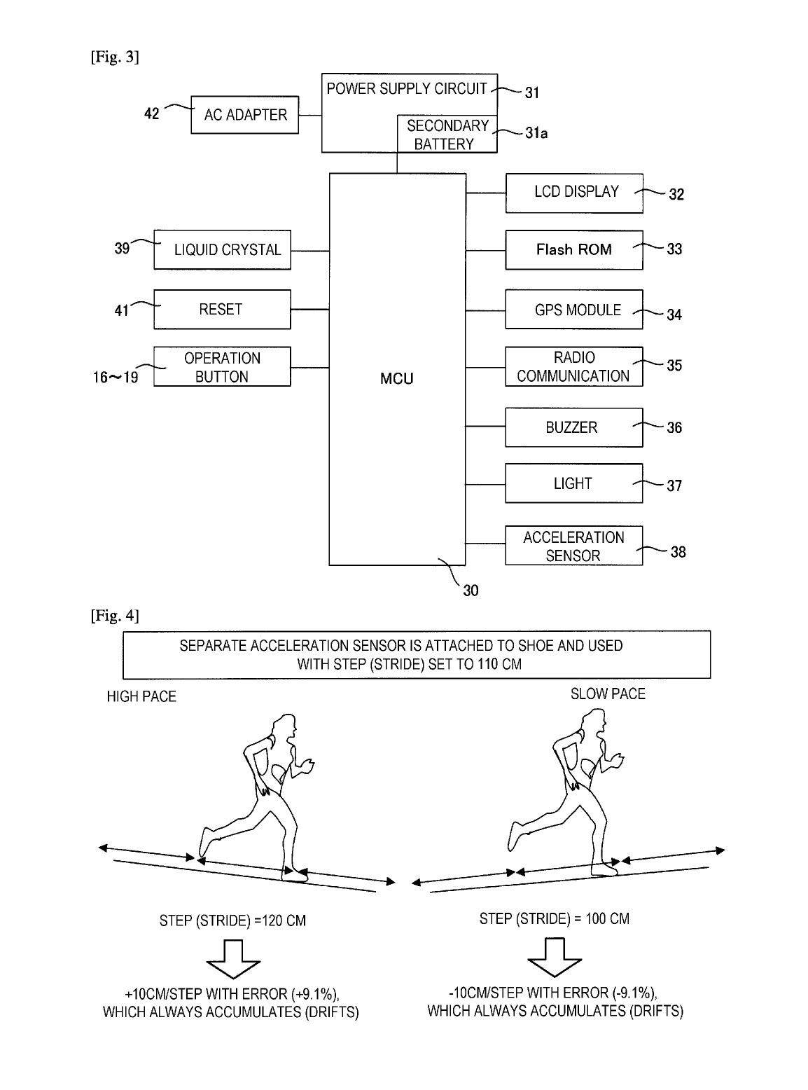

FIG. 3 is a block diagram showing a circuit configuration of the GPS running watch 100. As shown in FIG. 3, the GPS running watch 100 includes an MCU 30, a power supply circuit 31, a liquid-crystal-panel display unit 32, a flash ROM 33, a GPS module 34, a radio communication unit 35, a buzzer 36, a light 37, an acceleration sensor 38, a quartz oscillator 39, a reset circuit 41, and the operation buttons 16 to 19.

The MCU 30 includes, on the inside thereof, a memory configured to store a program. The MCU 30 is configured to perform control of the units of the GPS running watch 100 and perform storage processing for a running state of a user, speed calculation processing, and the like explained below. When connected to an AC adapter 42, the power supply circuit 31 charges a secondary battery 31a. The secondary battery 31a supplies driving power to the liquid-crystal-panel display unit 32, the GPS module 34, and the like.

The GPS module 34 performs processing for acquiring satellite information such as satellite orbit information, GPS time information, or position information included in a navigation message from a satellite signal in a 1.5 GHz band extracted by a not-shown SAW filter. For example, time difference information is stored in the flash ROM 33. The time difference information is information in which time difference data (e.g., a correction amount for UTC associated with a coordinate value (e.g., latitude and longitude) is defined. As explained below, a correlation between a body vibration frequency and speed is also recorded in the flash ROM 33.

The radio communication unit 35 is configured to be capable of performing radio communication between the GPS running watch 100 and a personal computer and the like and transmitting log data and the like stored in the GPS running watch 100 to the personal computer and the like. The buzzer 36 is used for, for example, informing completion of setting processing by the user. The light 37 is used to irradiate light on the liquid crystal panel 40 according to operation of an operation button by the user and facilitate visual recognition by the user at night and the like.

The acceleration sensor 38 is a sensor capable of detecting accelerations in three axis directions, i.e., an X-axis direction equivalent to the lateral direction, a Y-axis direction equivalent to the longitudinal direction, and a Z-axis direction equivalent to a direction perpendicular to the cover glass 82 of the GPS running watch 100 when the GPS running watch 100 is viewed from the front as shown in FIG. 2. That is, in a state in which a user wears the GPS running watch 100 on the arm and runs with the thumb facing upward, a running direction of the user is the X-axis direction, an up down moving direction of the user (the gravity direction) is the Y-axis direction, and a left right moving direction of the user is the Z-axis direction. The acceleration sensor 38 is explained in detail below. The quartz oscillator 39 is a quartz oscillation circuit attached with a temperature compensation circuit. The quartz oscillator 39 generates a reference clock signal having a substantially fixed frequency irrespective of temperature. The reset circuit 41 is used for resetting the operation of the GPS running watch 100. The operation buttons 16 to 19 are used to switch an operation mode (the running mode, the time display mode, etc.) and perform display setting for the liquid crystal panel 40 and various setting inputs.

### C: Speed Estimation Processing and Distance Calculation Processing Performed by Using the Acceleration Sensor of the GPS Running Watch

Speed estimation processing and distance calculation processing performed by using the acceleration sensor of the GPS running watch 100 according to this embodiment are explained in detail. For example, when radio wave information from the GPS satellites cannot be received or when radio waves from the GPS satellites are weak, speed information grasped by processing signals included in the radio wave information from the GPS satellites cannot be used.

As a method of estimating running speed when the speed information grasped by processing the signals included in the radio wave information from the GPS satellites in this way cannot be used, there is a method of attaching a separate device incorporating an acceleration sensor to a shoe of the user and measuring running speed using the device. However, in this method, fluctuation occurs in a measured value depending on setting of a step by the user. Even if the user is the same, a step is different when a running pace is a high pace and when the running pace is a slow pace. As shown in FIG. 4, for example, even if the step is set to 110 cm, the step sometimes increases to 120 cm during the high pace on a downhill road or the like and sometimes decreases to 100 cm during the slow pace on an uphill road or the like. Therefore, there is an error of plus-minus 10 cm per one step. Since the error always accumulates, in the method of using the separate acceleration sensor, it is difficult to accurately measure running speed.

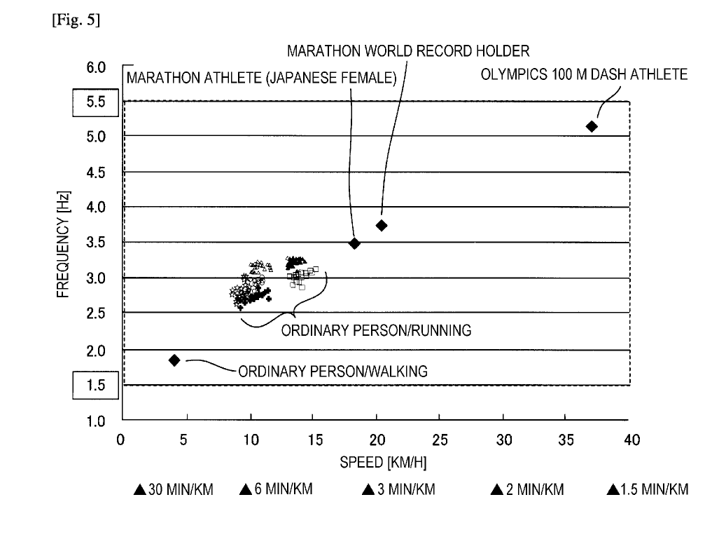

However, it has been found as a result of examination that a body vibration frequency, i.e., a frequency of vibration of the body of a runner moving up and down during running does not depend on the step of the runner and has a correlation with actual running speed of the runner. It has also been found that a relation between the body vibration frequency and the running speed does not depend on a difference of an exercise capacity of the runner either and, as shown in FIG. 5, is in a substantially proportional relation in runners ranging from general runners to Olympics athletes of field and track events and in a relation in which, as the body vibration frequency is higher, the running speed is higher. For example, it has been found that, as shown in FIG. 5, during running of a general runner, the body vibration frequency is about 2.5 Hz to 3.2 Hz, during running of an Olympics athlete of the marathon, the body vibration frequency is about 3.5 Hz to 3.7 Hz, and during running of an Olympics athlete of the 100 m dash, the body vibration frequency is about 5.1 Hz.

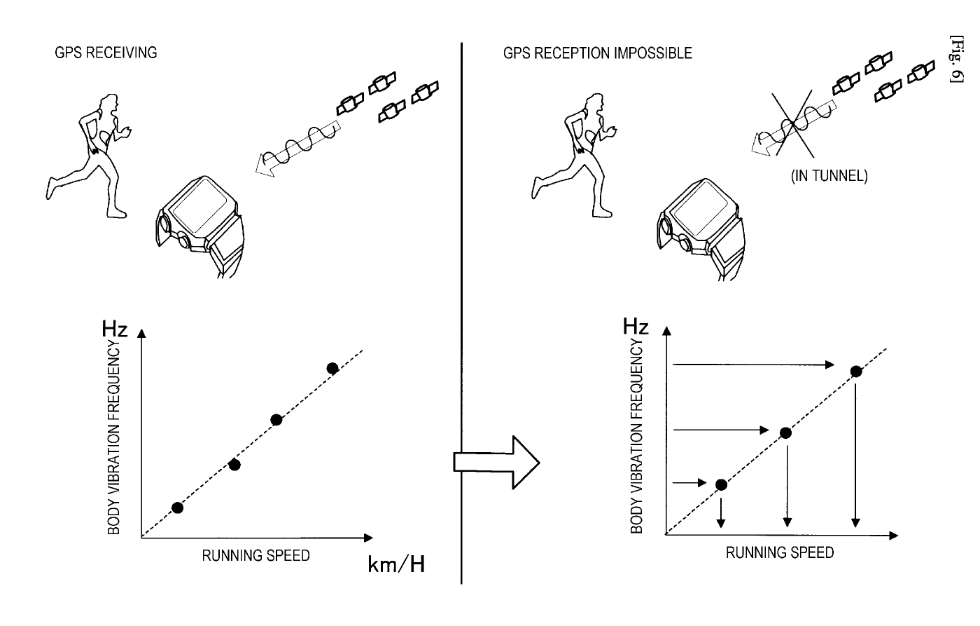

Therefore, in this embodiment, as an example, a frequency of vibration in the Y-axis direction of the acceleration sensor 38 is set as a body vibration frequency of the user and a correlation between the body vibration frequency and running speed is specified. Specifically, as shown in FIG. 6, when reception of radio wave information from the GPS satellites is satisfactorily performed, the body vibration frequency of the user is measured, speed information grasped by processing signals included in the radio wave information from the GPS satellites at that point is set as running speed, and data of the body vibration frequency of the user and data of the running speed are recorded in association with each other. A correlation between the body vibration frequency of the user and the running speed is specified as a linear expression. To specify the linear expression, for example, the method of least squares is used. When it is determined that the radio wave information from the GPS satellites cannot be satisfactorily received, the body vibration frequency of the user measured by the acceleration sensor is applied to the specified linear expression to estimate running speed and a distance is calculated from the speed.

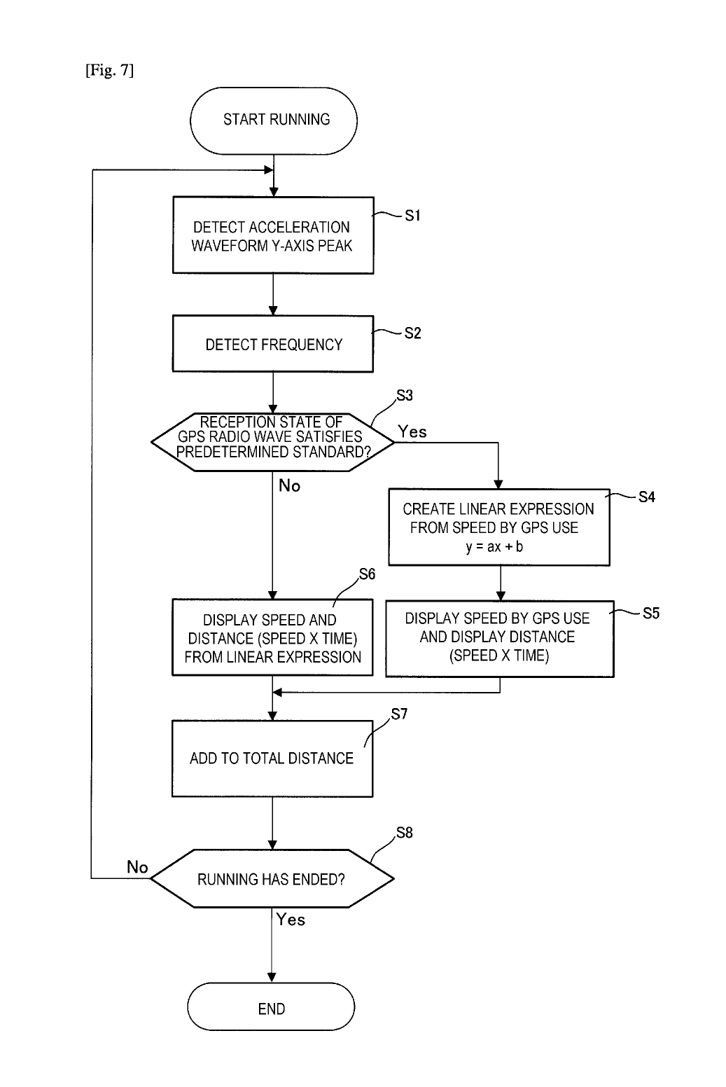

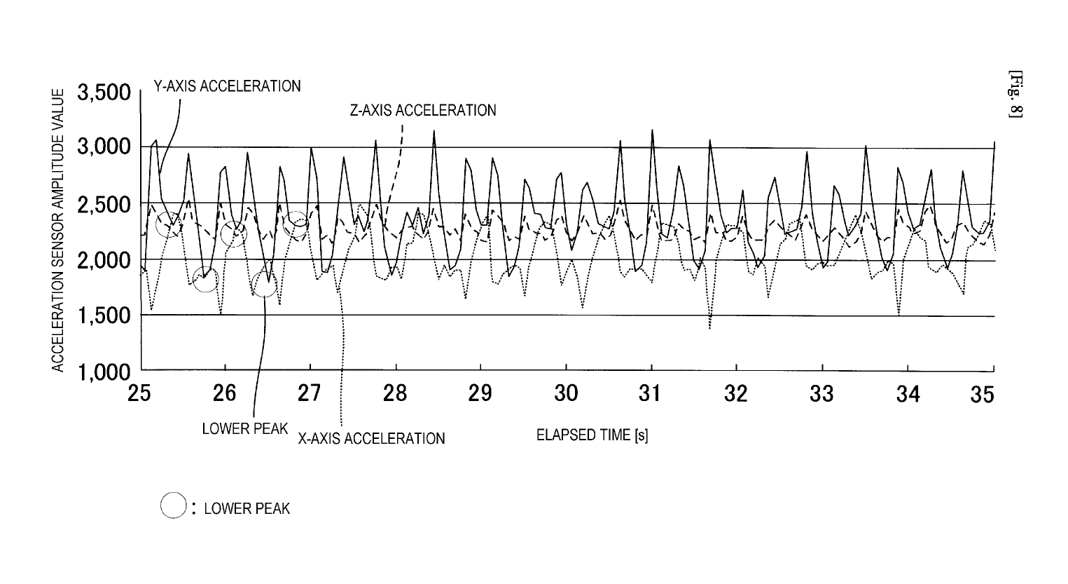

A specific example of the speed estimation processing and the distance calculation processing in this embodiment is explained on the basis of a flowchart of FIG. 7 and an output waveform chart of the acceleration sensor of FIG. 8. First, when starting running, the user operates the operation button 16 of the GPS running watch 100 to start measurement of speed and a distance. When the measurement is started, amplitude values of vibrations in the respective X-axis, Y-axis, and Z-axis directions are output from the acceleration sensor 38 as shown in FIG. 8. Therefore, as an example, the MCU 30 detects a lower peak value of an acceleration waveform in the Y-axis direction among acceleration waveforms output from the acceleration sensor 38 and stores, as required, time when the lower peak value is obtained (FIG. 7: S1).

The MCU 30 detects, from the time when the lower peak value is obtained, the number of vibrations in the Y-axis direction per hour, i.e., a frequency of vibration in the Y-axis direction and stores the frequency (FIG. 7: S2). Subsequently, the MCU 30 determines whether a reception state of radio waves from the GPS satellites satisfies a predetermined standard (FIG. 7: S3). When the reception state of the radio waves from the GPS satellites satisfies the predetermined standard (FIG. 7: S3: YES), the MCU 30 specifies a linear expression from the stored frequency and speed information grasped by processing signals from the GPS satellites at the time when the lower peak value is obtained (FIG. 7: S4).

The MCU 30 displays, as running speed, the speed information grasped by processing the signals from the GPS satellites at that time and multiplies the acquired speed information with time from the time when the speed information is acquired last time to the time when the speed information is acquired this time to calculate a running distance from the time when the speed information is acquired last time to the time when the speed information is acquired this time (FIG. 7: S5). The MCU 30 adds the calculated distance to the current total distance (FIG. 7: S7). The display of the total distance may be performed as required or may be performed according to operation of the operation button by the user.

Subsequently, the MCU 30 determines whether the running of the user has ended (FIG. 7: S8). When the user operates the operation button 16 of the GPS running watch 100 to end the measurement of speed and a distance, the MCU 30 only has to determine that the running has ended. When the running has not ended (FIG. 7: S8: NO), the MCU 30 repeats the processing explained above.

When the reception state of the radio waves from the GPS satellites does not satisfy the predetermined standard (FIG. 7: S3: NO), the MCU 30 applies the body vibration frequency obtained from the output of the acceleration sensor 38 to the calculated linear expression to calculate running speed and displays the running speed. The MCU 30 multiplies the calculated running speed with time from the time when the speed information is acquired last time or when the running speed is calculated last time to the time when the running speed is calculated this time to calculate a running distance from the time when the speed information is acquired last time or when the running speed is calculated last time to the time when the running speed is calculated this time and displays the running distance (FIG. 7: S6). The MCU 30 adds the calculated distance to the current total distance (FIG. 7: S7). The display of the total distance may be performed as required or may be performed according to operation of the operation button by the user.

Subsequently, the MCU 30 determines whether the running of the user has ended (FIG. 7: S8). When the user operates the operation button 16 of the GPS running watch 100 to end the measurement of speed and a distance, the MCU 30 may determine that the running has ended. When the running has not ended (FIG. 7: S8: NO), the MCU 30 repeats the processing explained above. The specified linear expression may be stored as it is even after the running has ended or may be cleared when another user uses the GPS running watch 100.

In FIG. 9, results obtained by recording data given by sets of body vibration frequencies and running speeds of various users and specifying linear expressions respectively from the recorded data are shown. As shown in FIG. 9, even if there is a difference in running speed depending on a user, relations between the body vibration frequencies and the running speed of the respective users can be represented by linear expressions.

Therefore, according to this embodiment, when the user runs in a section where the reception state of the radio waves from the GPS satellites does not satisfy the predetermined standard, for example, when the user runs in a tunnel or the like, it is possible to estimate running speed of the user on the basis of a linear expression specified while the reception state of the radio waves from the GPS satellites satisfies the predetermined standard. Therefore, the user can always check running speed of the user and can run at a desired pace or an appropriate pace.

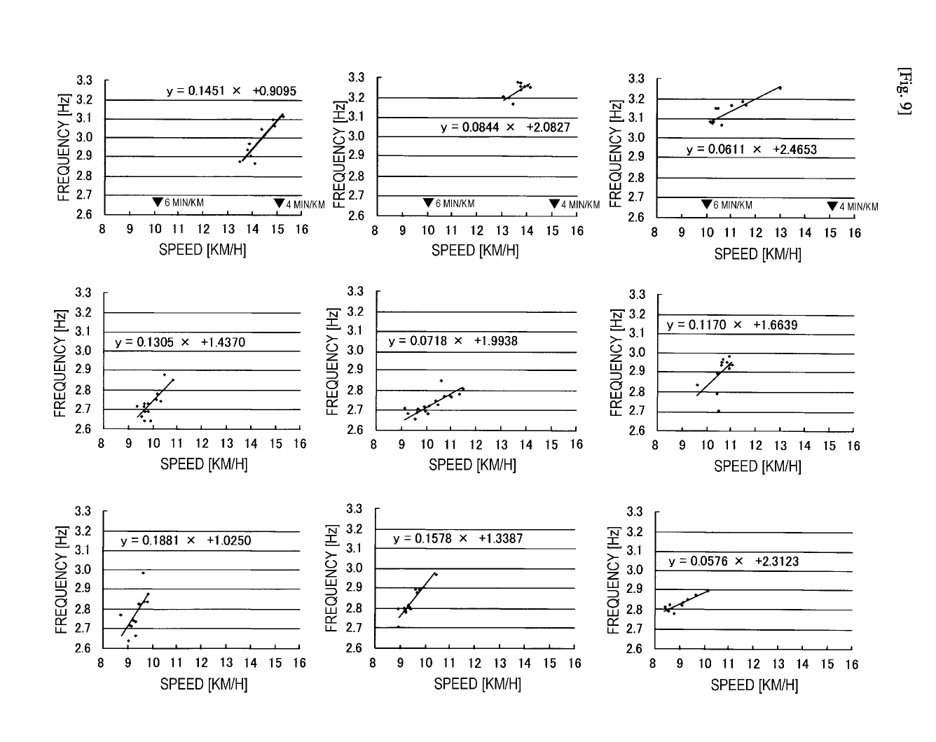

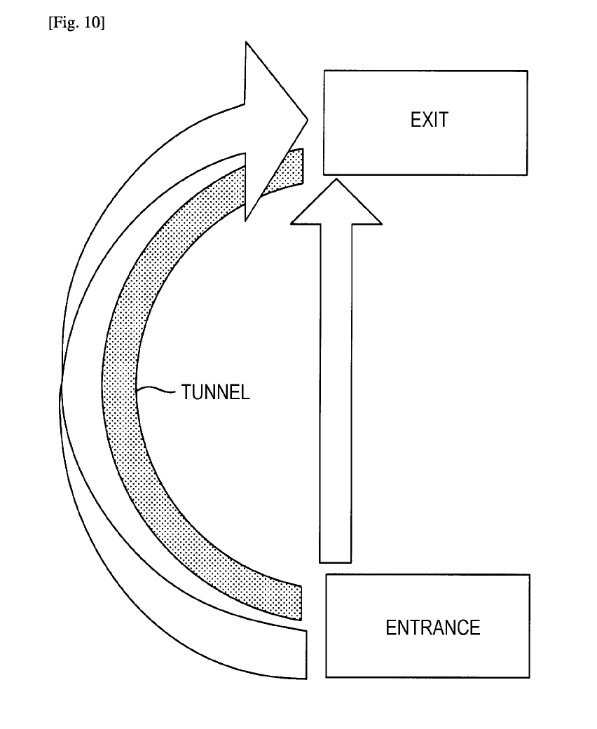

Further, according to this embodiment, not only while the reception state of the radio waves from the GPS satellites satisfies the predetermined standard but also when the user runs the section where the reception state of the radio waves from the GPS satellites does not satisfy the predetermined standard, the processing for calculating a running distance from estimated running speed and adding the running distance to the current total distance is performed. Therefore, for example, as shown in FIG. 10, even when the user runs in a curved tunnel, the user can accurately learn a running distance.

When the user runs in a curved tunnel shown in FIG. 10, if running speed and a running distance are determined at two measurement points, i.e., determined at a measurement point at the entrance of the tunnel where the reception state of the radio waves from the GPS satellites satisfies the predetermined standard and thereafter determined at a measurement point at the exit of the tunnel where the reception state of the radio waves from the GPS satellites satisfies the predetermined standard, wrong measurement is performed as if the user has run a straight tunnel.

However, in this embodiment, when it is determined that the user enters the tunnel and is running in a section where the reception state of the radio waves from the GPS satellites does not satisfy the predetermined standard, running speed and a running distance are calculated from the body vibration frequency measured by the acceleration sensor 38 and the specified linear expression. Therefore, even when the user runs in the curved tunnel shown in FIG. 10, the user can accurately learn the running distance.

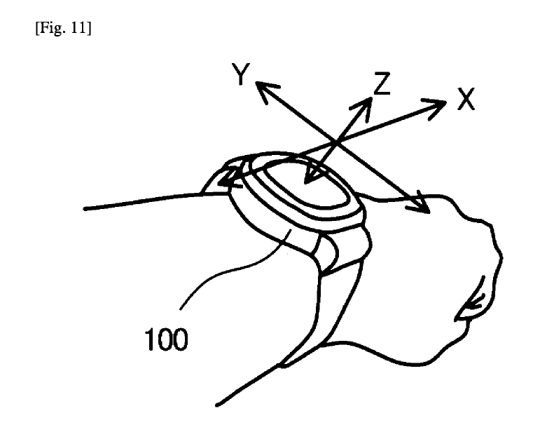

In this embodiment, the frequency of the vibration in the Y-axis direction of the acceleration sensor 38 is calculated and set as the body vibration frequency. However, the direction of the vibration to be set as the body vibration frequency only has to be a direction in which the body of the user moves up and down during running. Vibration of an axis closest to the gravity direction only has to be used as the vibration. The vibration of the axis closest to the gravity direction means vibration in an axis direction in which amplitude is the largest among vibrations in the X-axis direction, the Y-axis direction, and the Z-axis direction. For example, in a state in which the user wears the GPS running watch 100 on the arm and runs with the thumb facing upward, the amplitude of the vibration in the Y-axis direction shown in FIG. 11 is the largest. Therefore, the vibration in the Y-axis direction is used as the vibration of the axis closest to the gravity direction. However, as shown in FIG. 11, when the user attempts to look at the liquid crystal panel of the GPS running watch 100 while running, the amplitude of the vibration in the Z-axis direction, which is the direction perpendicular to the liquid crystal panel of the GPS running watch 100, is the largest. Therefore, in this case, the vibration in the Z-axis direction is used as the vibration of the axis closest to the gravity direction. An example of the vibrations in the respective axis directions is shown in FIG. 12.

As shown in FIG. 12, it is seen that, in the state in which the user wears the GPS running watch 100 on the arm and is running with the thumb facing upward, the amplitude of the vibration in the Y-axis direction is the largest. Therefore, it is seen that, when the user is looking at the screen of the GPS running watch 100, as shown in a region A of FIG. 12, the amplitude of the vibration in the Z-axis direction is the largest. In this embodiment, as explained above, the vibration in the axis direction in which the amplitude is the largest is adopted as the vibration of the axis closest to the gravity direction. A frequency of the vibration of the axis closest to the gravity direction is calculated and set as the body vibration frequency. However, the invention is not limited to such an example. A frequency of a waveform obtained by combining vibration waveforms of the axes of the acceleration sensor 38 may be set as the body vibration frequency.

In the example explained in the embodiment above, a linear expression is specified from the relation between the body vibration frequency and the running speed and the specified linear expression is stored. However, a table for specifying a correlation between the body vibration frequency and the running speed may be generated and stored. Running speed may be estimated using the table.

Whether the reception state of the radio waves of the GPS satellites satisfies the predetermined standard only has to be determined according to, for example, whether the number of receivable satellites decreases to be equal to or smaller than a predetermined number or whether an error of measurement positions increases to be equal to or larger than a predetermined value because of the satellites.

As explained above, according to this embodiment, the multi-axis acceleration sensor 38 is provided in the GPS running watch 100 to estimate running speed. Therefore, it is unnecessary to attach a sensor separate from the running watch to the foot or the chest. This makes it possible to facilitate preparation before running. The acceleration sensor 38 is incorporated in the GPS running watch 100. Therefore, there is no risk of a failure of data recording due to a dead battery or an old battery that occurs when the separate sensor is used.

In this embodiment, the motion of body vibration having large amplitude is detected. Therefore, even if the motions of the arms during running are various and complicated depending on users, it is possible to accurately catch a waveform peak and improve accuracy of an output value.

Further, some runner does not swing the arms when running. However, in this embodiment, since the motion of the body vibration having large amplitude is detected, even when such a runner who does not swing the arms uses the GPS running watch 100, it is possible to improve accuracy of an output value.

Since the user does not need to input a running distance before running, even when the user ran on an arbitrary route, it is possible to appropriately grasp a running distance of the running. Further, since it is unnecessary to set and input a step (a stride), it is possible to accurately calculate a running distance without being affected by a setting error of the step or an error of a step during running.

### D: Modifications

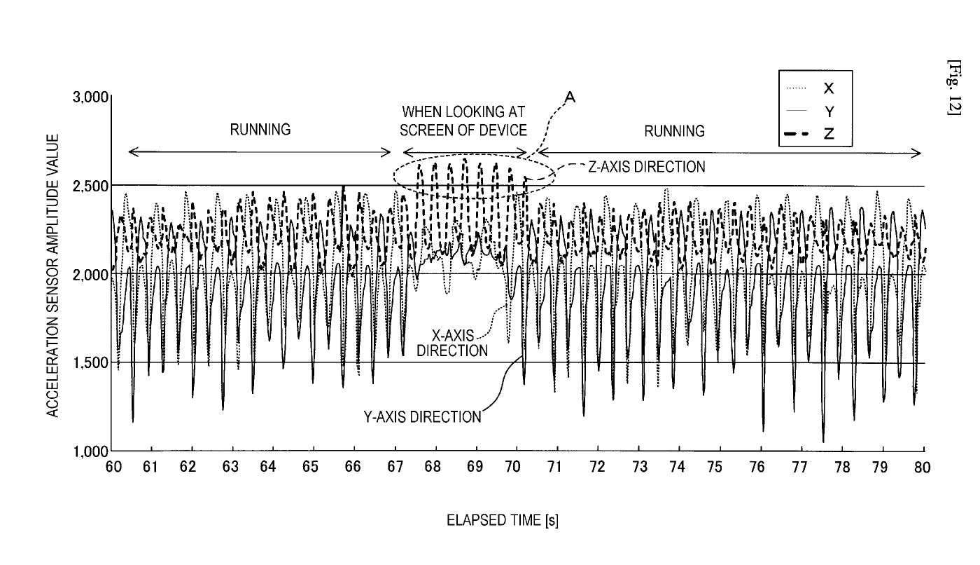

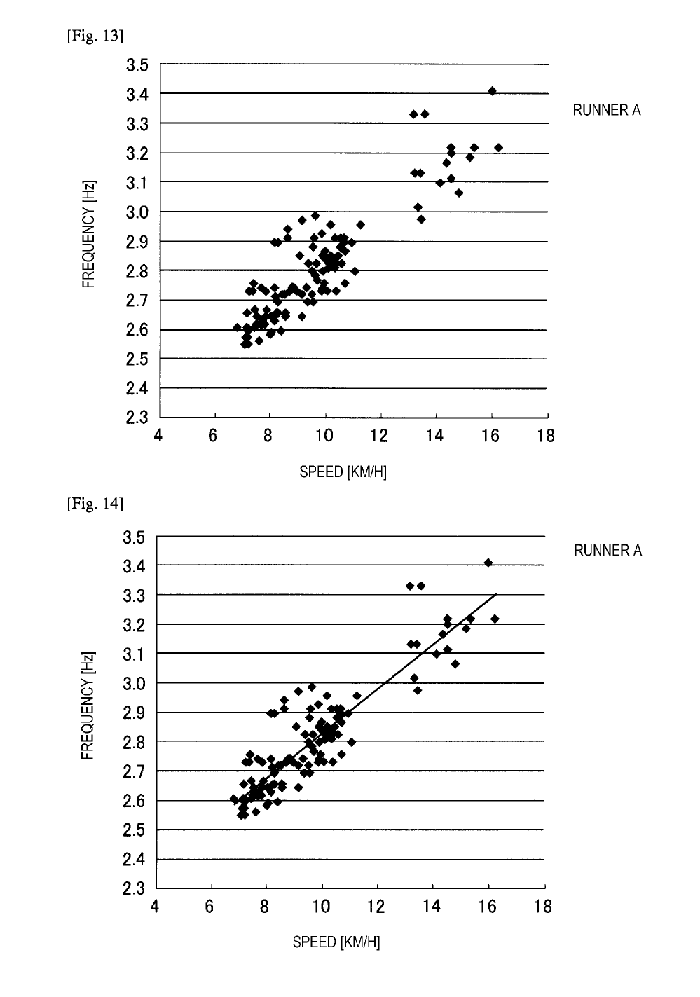

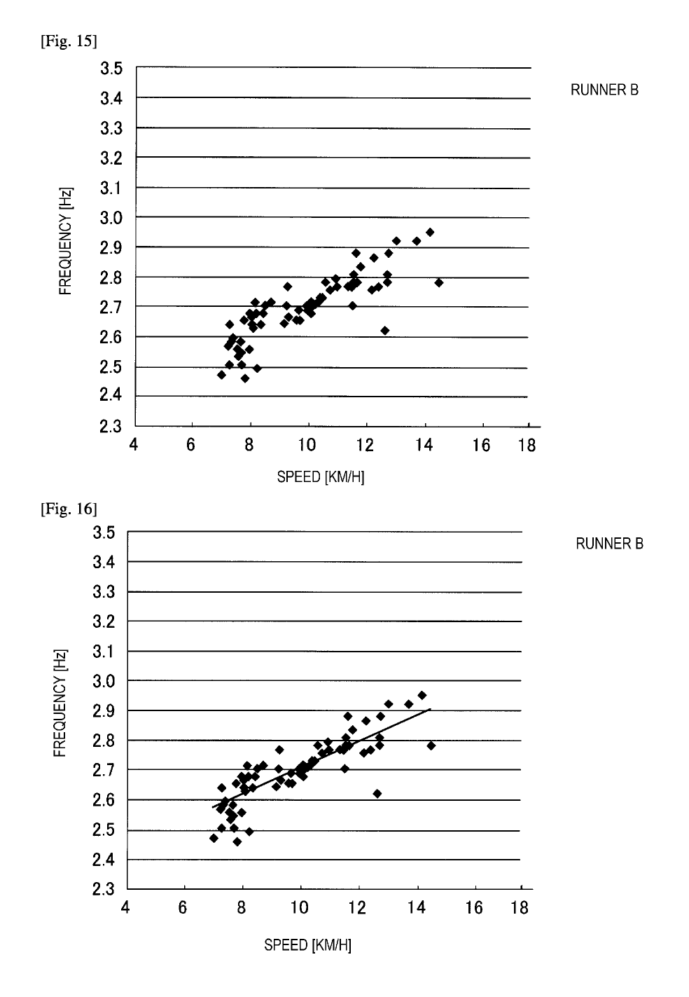

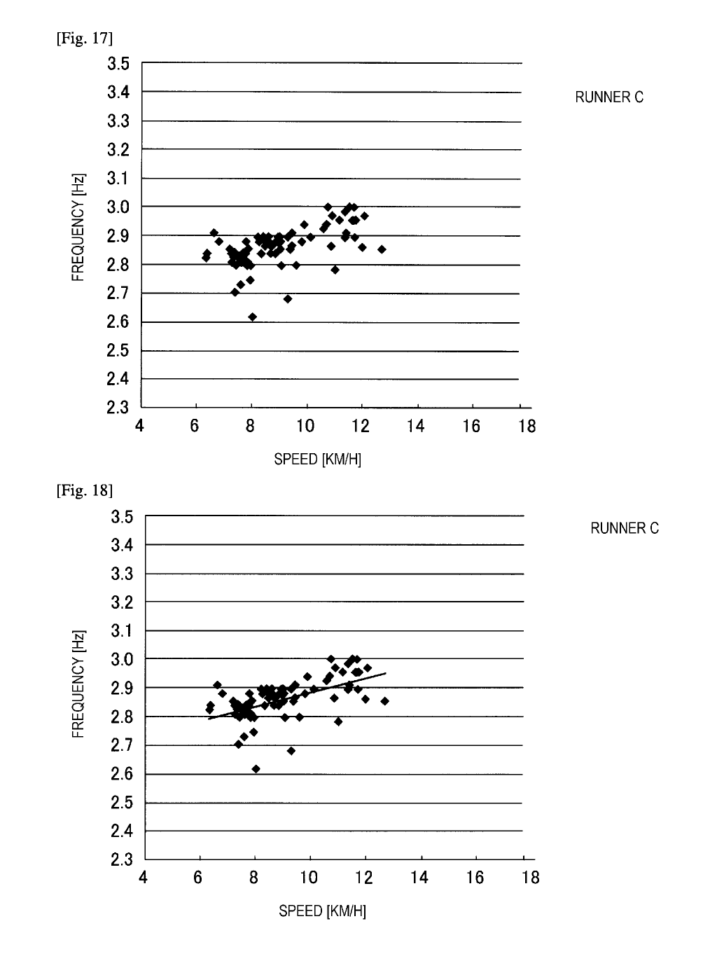

In the example explained in the embodiment above, a linear expression is calculated from the relation between the body vibration frequency and the running speed. However, a quadratic expression may be calculated. FIGS. 13, 15, and 17 are graphs respectively obtained by plotting data indicating relations between body vibration frequencies and running speeds of a runner A, a runner B, and a runner C. FIGS. 14, 16, and 18 are respectively examples in which linear expressions are calculated by the method of least squares on the basis of the data shown in FIGS. 13, 15, and 17.

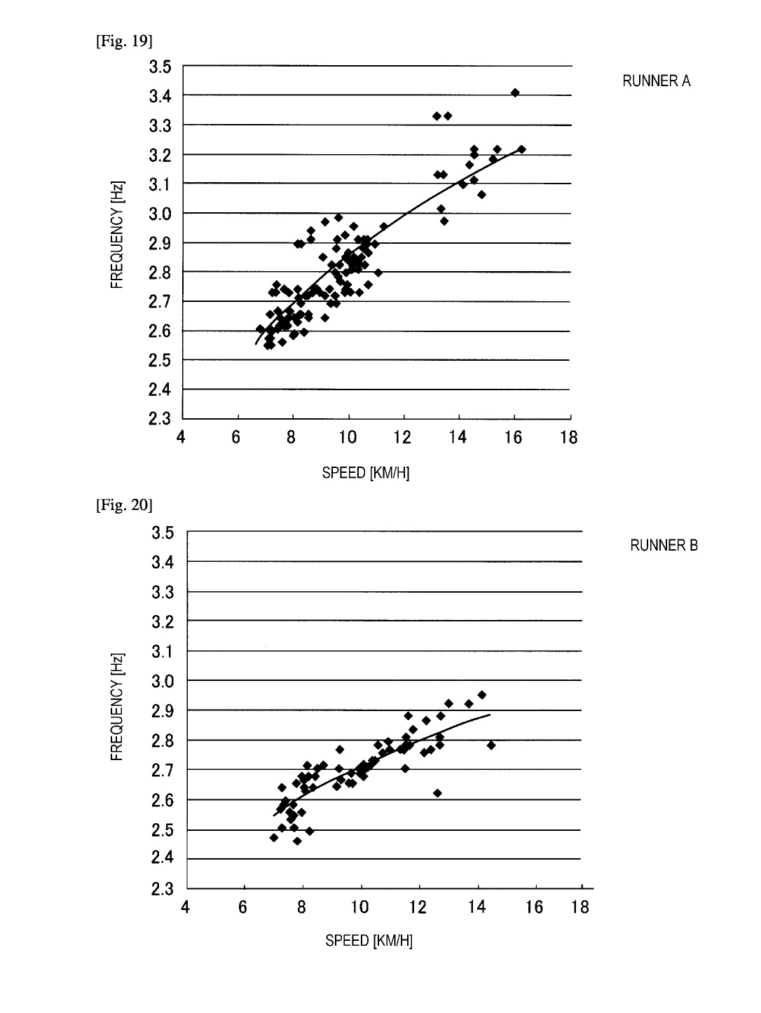

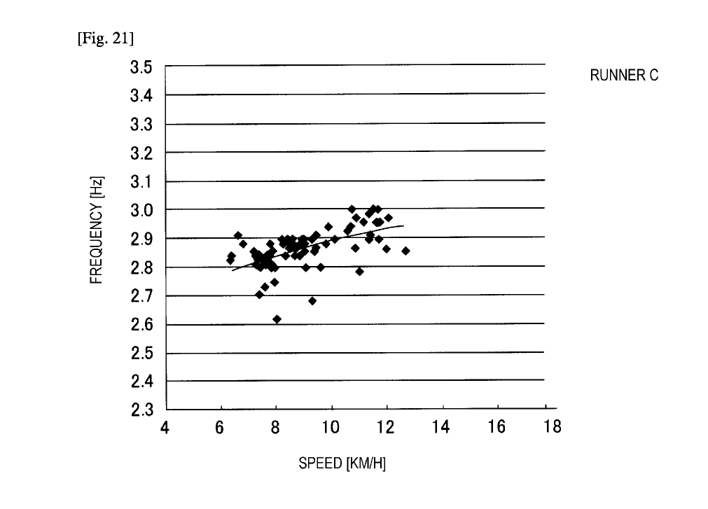

On the other hand, FIGS. 19, 20, and 21 are respectively examples in which quadratic expressions are calculated on the basis of the data shown in FIGS. 13, 15, and 17. In this way, according to this embodiment, it is possible to learn the relation between the body vibration frequency and the running speed as a quadratic expression. To specify the quadratic expression, the method of least squares only has to be used in the same manner as calculating the linear expression.

In the embodiment explained above, the GPS satellite 20 is explained as an example of the position information satellite included in the GPS system. However, this is only an example. The GPS system only has to be other global navigation satellite systems (GNSS) such as GALILEO (EU), GLONASS (Russia), and BEIDOU (China) or a GPS system such as the SBAS including position information satellites that transmit satellite signals such as stationary satellites and quasi-zenith satellites. That is, the GPS running watch 100 may be configured to acquire speed information grasped by processing radio waves (radio signals) from position information satellites including satellites other than the GPS satellites 20.

The speed information may be speed information itself included in radio waves from the position information satellites or may be information concerning groundspeed calculated from a running distance (a moving distance) and an elapsed time obtained by performing a positioning calculation using GPS time information and satellite orbit information included in the radio waves from the position information satellites.

Further, in the examples explained in the embodiment and the modifications, the correlation between the body vibration frequency and the running speed is specified. However, a correlation between the body vibration frequency and the running pace may be specified. The running pace may be specified from the specified correlation between the body vibration frequency and the running speed and the measured body vibration frequency. The running pace is an inverse of the running speed and only has to be represented by time (minutes) per 1 km. However, the running pace is not limited to such an example and only has to be represented by time (seconds, minutes, or hours) per a predetermined distance.

1. A portable device comprising:
a radio-wave receiving unit configured to receive a radio wave from a position information satellite;
a detecting unit capable of detecting a body vibration frequency in a direction in which a body of a user moves up and down during running;
a display unit configured to display running information;
a specifying unit configured to specify, when a reception state of the radio wave satisfies a predetermined standard, a correlation between the body vibration frequency detected by the detecting unit and speed information grasped by processing a signal included in the radio wave; and
an estimating unit configured to estimate, when the reception state of the radio wave does not satisfy the predetermined standard, running speed or a running pace on the basis of the body vibration frequency detected by the detecting unit and the correlation specified by the specifying unit.

2. The portable device according to claim 1, wherein the detecting unit includes an acceleration sensor configured to detect accelerations in three axis directions of an X axis, a Y axis, and a Z axis and detects, as the body vibration frequency, a frequency of vibration in a direction of an axis closest to a gravity direction among frequencies of vibrations obtained by the acceleration sensor.

3. The portable device according to claim 1, wherein the detecting unit includes an acceleration sensor configured to detect accelerations in three axis directions of an X axis, a Y axis, and a Z axis, combines waveforms of vibrations of the respective axes obtained by the acceleration sensor, and detects a frequency of a combined waveform as the body vibration frequency.

4. The portable device according to claim 1, wherein the detecting unit includes an acceleration sensor configured to detect accelerations in three axis directions of an X axis, a Y axis, and a Z axis and detects, as the body vibration frequency, a frequency of a waveform of an axis having largest amplitude among waveforms of vibrations of the respective axes obtained by the acceleration sensor.

5. The portable device according to claim 1, wherein the specifying unit specifies, as a linear expression, the correlation between the body vibration frequency and the speed information grasped by processing the signal included in the radio wave, and the estimating unit estimates running speed or a running pace from the body vibration frequency on the basis of the linear expression.

6. The portable device according to claim 1, wherein the specifying unit generates a table for specifying the correlation between the body vibration frequency and the speed information grasped by processing the signal included in the radio wave, and the estimating unit estimates running speed or a running pace from the body vibration frequency referring to the table.

7. The portable device according to claim 1, further comprising a running-distance calculating unit configured to calculate, when the reception state of the radio wave satisfies the predetermined standard, a running distance on the basis of the speed information and time grasped by processing the signal included in the radio wave and calculate, when the reception state of the radio wave does not satisfy the predetermined standard, the running distance on the basis of the running speed and time or the running pace and time estimated by the estimating unit, wherein the display unit displays the calculated running distance.

8. The portable device according to claim 1, further comprising a running-distance calculating unit configured to calculate, when the reception state of the radio wave satisfies the predetermined standard, a running distance on the basis of the position information grasped by processing the signal included in the radio wave and calculate, when the reception state of the radio wave does not satisfy the predetermined standard, the running distance on the basis of the running speed and time or the running pace or time estimated by the estimating unit, wherein the display unit displays the calculated running distance.
